(19) United States
(12) Patent Application Publication
(10) Pub. No.: US 2010/0086613 A1
(43) Pub. Date: Apr. 8, 2010

(54) CHITOSAN VEHICLE AND METHOD FOR MAKING SAME

(76) Inventors: Chang-Jer WU, Taipei (TW); Han-Ning Huang, Taipei (TW); Chien-Lung Chen, Tainan (TW); Cheng-Hsien Chen, Taipei (TW)

Correspondence Address: LAW OFFICES OF KHALILIAN SIRA, LLC, 9100 PERSIMMON TREE ROAD, POTOMAC, MD, 20854, US

(21) Appl. No.: 12/245,602

(22) Filed: Oct. 3, 2008

(51) Int. Cl. A61K 9/14

(52) U.S. Cl. 424/499

(57) ABSTRACT

The invention relates to chitosan (CS) vehicles with chitosan nanoparticles, as well as methods for making such chitosan vehicles and for using them to carry a DNA or proteins by forming CS-DNA or CS-protein complexes. The present invention also relates to CS-DNA or CS-protein complexes being useful for transdermal delivery of DNA or protein with a low-pressure gene gun. In another aspect, the present invention also relates to CS-DNA or CS-protein complexes being useful for transcutaneous delivery of a DNA or protein with a skin patch. Further aspects of the present invention relate to methods for making CS-DNA or CS-protein complexes and for using them for diagnostic, therapeutic and biological industrial applications.

### BACKGROUND OF THE INVENTION

1. Field of the Invention

The present invention relates to a chitosan vehicle, especially to a chitosan vehicle comprising chitosan nanoparticles.

2. Description of the Prior Art

DNA vaccines have most frequently been delivered either by injection into muscle tissue or by particle bombardment (gene gun) to dermis (Chen et al., J Virol 73:10137-45 (1999)). However, DNA-coated gold particles should be bombarded directly into the cytoplasm and nuclei of cells facilitating expression of the encoded protein. So, gene guns usually use the high-pressure helium to deliver the DNA-coated gold particles through the stratum corneum of the epidermis (Bellhouse et al., U.S. Pat. No. 5,899,880 (1999)).

In addition, the high-pressure gene gun has some disadvantages. Gene gun bombardments is often considered to be too expensive, requires skillful operators and its uses non-biodegradable gold particles. The non-biodegradable gold particles used with a conventional gene gun may cause adverse side effects should they accumulated in the body (Lin et al., Mol Ther 13:S291 (2006)).

Until recently, transcutaneous immunization (TCI) was the primary route for administration of a DNA vaccine. Needle-free vaccination approaches have recently been under intensive investigation because they are convenient, easily accepted by patients and relatively safe. A promising vaccination approach is the transcutaneous delivery of antigen in the form of a skin patch, as the skin is a potent immunological site with abundant antigen presenting cells. Transcutaneous immunization (TCI) is also capable of inducing broader immune responses because it targets the Langerhans and dermal dendritic cells, which reside in the epidermis and dermis (Romani et al., J Exp Med 161(6):1368-83 (1985); Berry et al., Infect Immun 72(2):1019-28 (2004)).

In several reports, these adjuvants or their subunits were used with patches or other methods to elicit robust immune responses against coadministered antigens via skin delivery. The most commonly used adjuvants are cholera toxin (CT), pseudomonas exotoxin A, diptheria toxin and heat-labile enterotoxin (LT) from Escherichia coli, their subunits and their mutants (Kersten et al., Infect Immun; 68(9):5306-13 (2000)). These adjuvants were reported to trigger systemic and mucosal antibody responses against the toxin and the co-administered antigens as well as antigen-specific T cell responses (Freytag et al., Curr Top Microbiol Immunol 236:215-36 (1999)). But such treatments were less acceptable for future clinical use.

To overcome the shortcomings of conventional techniques as described above, the present invention provides a chitosan vehicle and a method for making the same to mitigate or obviate the aforementioned problems.

### SUMMARY OF THE INVENTION

The main objective of the invention is to provide chitosan vehicles comprising chitosan nanoparticles, as well as methods for making such chitosan vehicles and use of chitosan vehicles to carry DNA (deoxyribonucleic acid) or proteins by forming CS-DNA or CS-protein complexes.

The present invention also relates to CS-DNA complexes being used for transdermal delivery of DNA with a low-pressure gene gun, as well as CS-DNA complexes being implemented in transcutancous delivery of DNA with a skin patch. The CS-DNA complexes are specifically useful for delivery of an immunogenic DNA such as a DNA vaccine.

In another aspect, the present invention also relates to CS-protein complexes being used for transdermal delivery of a protein with a low-pressure gene gun, as well as CS-protein complexes implemented for transcutaneous delivery of a protein with a skin patch. The CS-protein complexes are specifically useful for delivery of a protein, such as collagen, for aesthetic, reconstructive or cosmetic purposes.

Other objectives, advantages and novel features of the invention will become more apparent from the following detailed description when taken in conjunction with the accompanying drawings.

### BRIEF DESCRIPTION OF THE DRAWINGS

FIG. 1 is a chart showing the mean particle size and zeta potential on the various N/P ratios of CS-DNA complex nanoparticles.

FIG. 2 is a chart showing the expression of JEV envelope protein at the skin with transdermally delivered CS/pCJ-3/ME complexes by low pressure gene gun.

FIG. 3A is a chart showing anti-E antibodies of C3H/HeN mice immunized with transdermally delivered CS/pCJ-3/ME complexes by low pressure gene gun.

FIG. 3B is a chart showing protective effects of C3H/HeN mice immunized with transdermally delivered CS/pCJ-3/ME complexes by low pressure gene gun.

FIG. 4 is a chart showing the effects of the ratio of DNA to CS on transfection efficiency of BHK-21 cell and Vero cell.

FIG. 5 is a chart showing the expression of JEV envelope protein at the skin with transcutaneously delivered CS/pCJ-3/ME complexes by skin patch.

FIG. 6A is a chart showing anti-E antibodies of C3H/HeN mice immunized with transcutaneously delivered CS/pCJ-3/ME complexes by skin patch.

FIG. 6B is a chart showing protective effects of C3H/HeN mice immunized with transcutaneously delivered CS/pCJ-3/ME complexes by skin patch.

FIG. 7 is a chart showing the cell viability of BHK-21 cells treated with CS-protein complexes (CS-collagen complexes).

FIG. 8 is a chart showing the nitrite accumulation of RAW 264.7 cells after treatment with different concentration of CS-collagen complexes.

FIG. 9 is a chart showing the fluorescence intensity of FITC-labeled CS-collagen complexes transdermally delivered by a low-pressure gene gun and transcutaneously delivered by skin patch.

### DETAILED DESCRIPTION OF THE PREFERRED EMBODIMENTS

### Chitosan Vehicle

The present invention provides chitosan vehicles comprising chitosan nanoparticles. The chitosan nanoparticles are made from a chitosan being at least 81% deacetylated. Highly deacetylated chitosan is known to be less toxic to an individual, either human or animal. Preferably, the chitosan for making the nanoparticles is 81-85% deacetylated.

The molecular weight of the chitosan is not specifically limited in the present invention. A chitosan of molecular weight ranging from 15 kDa to 800 kDa is acceptable for making the aforementioned chitosan nanoparticles of the chitosan vehicle, wherein a molecular weight ranging from 80 to 108 kDa is preferred. The chitosan may be obtained from a natural source and may be provided by commercial sources. One example of a natural source is crustacean shells while an example of a commercial source is a chemical provider such as Sigma (St. Louis, Mo.).

The Chitosan vehicle is useful for carrying a DNA or proteins for transdermal or transcutaneous delivery in applications in treating an individual. Examples of the DNA include but are not limited to a gene-therapeutic DNA, plasmids and an immunogenic DNA such as a DNA vaccine. The proteins may be antigens, antibodies, antibiotic agents or a cosmetic protein (such as but not limited to collagen). The individual may be human or a non-human mammalian in need of treatment with the aforementioned DNA or proteins. One example of such treatment is immunogenicity against a pathogen such as Japanese encephalitis virus (JEV).

### Method for Making Chitosan Vehicles

A method for making the chitosan vehicle comprises dissolving a chitosan in an acetic acid solution to generate a chitosan solution; adjusting the chitosan solution to pH 4.0-6.0; and degrading the chitosan solution to generate chitosan nanoparticles having an average diameter of 200 nm. Preferably, the acetic acid solution is a 167 mM acetic acid solution and the chitosan is dissolving in the acetic acid solution to a concentration of 1% (w/v). Furthermore, it is preferred that the chitosan is at least 81% deacetylated. More preferably, the chitosan is 81-85% deacetylated,

A preferred method of making the chitosan vehicles comprises (a) dissolving the aforementioned chitosan of the chitosan vehicle to a concentration of 1% (w/v) in 167 mM acetic acid solution under gentle heat to generate a chitosan solution; (b) adjusting the chitosan solution to pH 5.5-6.0; and (c) degrading the chitosan solution with methods such as ionic-gelating the mixture by ultrasonication at room temperature; (d) centrifuging the chitosan solution at 12,000 g and removing pelleted particles to generate chitosan nanoparticles. As one of ordinary skill would appreciate, examples of a method for degrading the chitosan solution to generate nanoparticles include but are not limited to ionic gelation, emulsion polymerization and emulsification-diffusion.

### CS-DNA (Chitosan-Deoxyribonucleic Acid) Complex

The present invention also relates to a CS-DNA complex useful for transdermal or transcutaneous delivery of DNA in an individual and comprises a chitosan vehicle having chitosan nanoparticles, a crosslinking agent and a DNA crosslinked to the chitosan nanoparticles of the chitosan vehicle. In a preferred embodiment of the present invention, the CS-DNA complex is prepared according to methods previously reported (Sato, et al., Chem Lett 1996:725-6 (1996)). One of such method comprises preparing aqueous solution of a sonicated DNA; mixing the DNA solution with polysaccharide solutions, wherein the molar ratios of the phosphate anion of DNA to the cation of the polysaccharides are 1:2; allowing interaction of the DNA and the polysaccharide; obtaining a polysaccharide-DNA complex. In the present invention, it is preferred that the chitosan nanoparticles of the chitosan vehicle is made from at least 81% deacetylated chitosan. More preferably, the chitosan vehicle is made from 81-85% deacetylated chitosan.

The CS-DNA complex comprises the chitosan vehicle, a crosslinking agent such as TPP (thiamine pyrophosphate) and a DNA being a DNA plasmid being crosslinked to the chitosan nanoparticles of the chitosan vehicle by the crosslinking agent. In a preferred embodiment, 8 ml of a 0.84 mg/ml TPP aqueous solution is added into 20 ml of the chitosan solution. As aforementioned, the chitosan nanoparticles of the chitosan vehicle is preferred to be made from at least 81% deacetylated chitosan. More preferably, the chitosan vehicle is made from 81-85% deacetylated chitosan.

The CS-DNA complex is useful for transdermal delivery of the DNA into an individual with a low-pressure gene gun. A low-pressure gene gun is more preferable than a conventional high-pressure gene gun for being less harmful to the individual. However, compared with metal nanoparticles used with a high-pressure gene gun, the great merit of the CS-DNA complex in accordance with the present invention is its biodegradable nature. With the CS-DNA complex and the low-pressure gene gun, one is able to efficiently deliver DNA transdermally into an individual. In addition, the CS-DNA complex is also useful for transcutaneous delivery of DNA into an individual with a skin patch comprising the CS-DNA complex. With the CS-DNA complex provided, transcutaneous delivery of DNA can be carried out efficiently without adjuvants. The CS-DNA complex may also be used as an ointment applied directly to skin.

The DNA plasmid comprises phosphate anions and the chitosan nanoparticles comprise amino groups. Preferably, the ratio (abbreviated as “N/P ratio”) of the phosphate anions (abbreviated as “P”) to the amino groups (abbreviated as “N”) ranges from 1:0.25 to 1:5. Mixing 1:1 chitosan/DNA plasmid results in precipitate. A mixture of 1:1 chitosan/DNA plasmid is not used in the present invention. A preferred N/P ratio ranges from 1:2 to 1:5.

### Method for Making CS-DNA Complexes

The CS-DNA complex is primarily made by crosslinking DNA and the chitosan nanoparticles of the aforementioned chitosan vehicle. A method for making CS-DNA complexes comprises (a) preparing a chitosan vehicle having chitosan nanoparticles; (b) mixing a DNA with the chitosan vehicle; and (c) adding a crosslinking agent such as TPP allowing the DNA to be crosslinked to the chitosan nanoparticles.

Preferably, in step (a), the chitosan nanoparticles of the chitosan vehicle is made from a chitosan solution of pH 5.5 and at least 81% deacetylated chitosan. More preferably, the chitosan vehicle is made from 81-85% deacetylated chitosan.

The DNA preparing process depends largely on the structure and features of DNA. In a preferred embodiment of the present invention, the CS-DNA complex comprises chitosan/DNA plasmid complex nanoparticles. One example of the method for making chitosan/DNA plasmid complex nanoparticles comprises mixing 10 μl of plasmid DNA solution (1 mg/ml) with the chitosan nanoparticles of the chitosan vehicle. The mixtures are gently stirred at room temperature for a couple of hours, allowing the DNA to be crosslinked to the chitosan nanoparticles.

### DNA

The DNA used to form the CS-DNA complex may be gene-therapeutic DNA, plasmids and an immunogenic DNA such as a DNA vaccine An example of the DNA is a JEV DNA vaccine prepared from a JEV ME protein expression cDNA. To generate the Beijing-1 JEV ME protein expression construct, cDNA coding for the ME gene (bases 431-2477) was obtained by reverse transcription and PCR amplification of Beijing-1 JEV genomic RNA, and cloned into the HindIII/EcoRI sites in the multiple cloning site of plasmid pCS-3 to produce plasmid pCJ-3/ME (Wu et al., Microbes Infect 8: 2578-2586 (2006)). Plasmid DNA was purified from transformed E. coli DH5α using a Qiagen Plasmid Giga kit (Qiagen, Hilden, Germany) according to the manufacturer's instructions, and stored at −70° C. as pellets. For use, it was dissolved in water at a concentration of 1 mg/ml.

Another example of the DNA is a plasmid comprising at least one reporter gene. Such a DNA, being a plasmid, is prepared from a pGFP-N1 vector and a pCMVβ vector (Clontech, Palo Alto, Calif.). The pGFP-N1 vector contains a green fluorescent protein and pCMVβ vector contains a β-galactosidase gene driven by a cytomegalovirus promoter. The DNA is amplified in E. coli DH5α (Life Technologies, Gaithersburg, Md.) by a standard procedure and purified. The DNA may be purified using a plasmid purification kit such as Plasmid Mega (Qiagen, Hilden, German). The DNA recovered is stored at 4° C. in sterile deionized (DI) water.

### Adjuvant

In an embodiment of the CS-DNA complex comprising a DNA vaccine crosslinked to chitosan nanoparticles. Preferably, the CS-DNA complex further comprises an adjuvant. The adjuvant stimulates the immune system of an individual to increased immunization effects induced by the DNA vaccine. A skilled artisan of the field would appreciate the skills relates to the selection of available adjuvants. One example of such adjuvant is an ODN containing CpG motif.

### CS-Protein Complex

The present invention is also directed to CS-protein complexes being useful for transdermal or transcutaneous delivery of proteins. Chitosan nanoparticles were produced by ionic gelation of the positively charged CS with the TPP. The protein stock solution was premixed with different volume chitosan nanoparticles. The ratio (abbreviated as “P/C ratio”) of the protein volume (abbreviated as “P”) to the chitosan nanoparticles volume (abbreviated as “C”) ranges from 1:0.5 to 1:5. A preferred P/C ratio is 1:2. The ratio is under magnetic stirring at room temperature.

The CS-protein complex is useful for transdermal delivery of the protein into an individual with a low-pressure gene gun being less harmful to the individual. The CS-protein complex is also beneficial due to its biodegradable nature. With the CS-protein complex and the low-pressure gene gun, efficient transdermal delivery of the protein into an individual is possible. In addition, the CS-protein complex is also useful for transcutaneous delivery of the protein into an individual without adjuvants. The CS-protein complex may also be used as a skin patch or an ointment being applied directly to skin.

### Method for Making CS-Protein Complexes

A method for making CS-protein complexes comprises: (a) dissolving a chitosan to an acetic acid solution under gentle heat to generate a chitosan solution; (b) adjusting the chitosan solution to pH 6; (c) degrading the chitosan solution to generate chitosan nanoparticles in the chitosan solution; (d) centrifuging the chitosan solution at 12,000 g and removing pelleted particles; (e) diluting the chitosan nanoparticles solution with water to make an aqueous chitosan solution; (f) preparing a protein stock solution comprising a protein; (g) mixing the protein stock solution with the aqueous chitosan solution at room temperature to generate a mixture solution comprising a mixture of the protein and chitosan nanoparticles; (h) allowing the mixture solution to react for a period of time at room temperature; (i) centrifuging the mixture solution to generate a supernatant having a CS-protein complex; (j) obtaining the CS-protein complex.

Preferably, in step (a) the acetic acid solution is a 167 mM acetic acid solution. In step (c), a method for degrading the chitosan solution may be ionic-gelating the mixture under ultrasonication at room temperature. In step (g), it is preferred to mix the protein stock solution with the aqueous chitosan solution under magnetic stirring. In step (h), the period of time is preferred to be 2 hours. In step (i), the mixture solution may be centrifuged at 14,000 rpm for 30 minutes. Furthermore, it is preferred that the chitosan in step (a) is at least 81% deacetylated. More preferably, the chitosan in step (a) is 81-85% deacetylated.

In a preferred embodiment of the method, in step (f), the P/C ratio, i.e. the ratio of the protein to the chitosan nanoparticles, is 1:2. In step (c), a 29 W ultrasonication machine may be employed and the 10 mg/ml chitosan solution is allowed to be ultrasonicated for 4 minutes.

In another preferred embodiment of the method, in steps (e) to (g), the aqueous chitosan solution is diluted with DI water to 50 μg/mL and the protein stock solution is a 1 mg/mL protein solution. Furthermore, 1 mL of the 1 mg/mL protein stock solution is mixed with 2 mL of the 50 μg/mL aqueous chitosan solution.

### Protein

The protein used to form the CS-protein complex may be an antibody, an antigen, an antibiotic agent, a protein for aesthetic, reconstructive or cosmetic purpose or a protein for skin repair. An example of an aesthetic, reconstructive or cosmetic protein is collagen for improving skin status and providing aesthetic or cosmetic appearances. An example of a protein for skin repair is a growth factor such as EGF (epidermal growth factor). The term protein as used herein also includes peptides.

### Application

In an application aspect, the present invention provides a gene gun projectile comprising the CS-DNA complexes or the CS-protein complexes, a skin patch comprising the CS-DNA complexes or the CS-protein complexes, as well as an ointment comprising the CS-DNA complexes or the CS-protein complexes.

The aforementioned gene gun projectile, skin patch and ointment are useful in biological industrial applications and may also be useful in fields related to diagnosis and therapy.

### EXAMPLES

### Example 1

### Immunization Against JEV by DNA Vaccine Transdermally Delivered with Low-Pressure Gene Gun

### Preparation of Nanoparticles

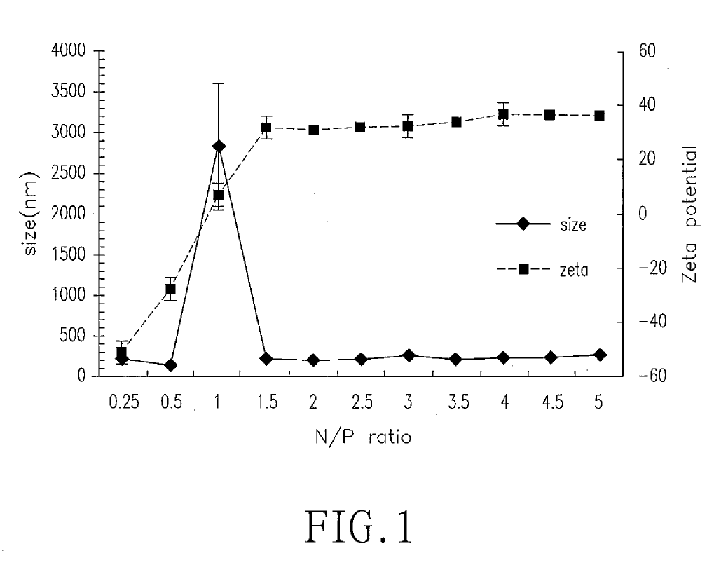

With reference to FIG. 1, chitosan nanoparticles were produced by ionic gelation of the positively charged CS with the TPP. The particle size and zeta potential of nanoparticles prepared at varying N/P molar ratios are shown in FIG. 1. The CS:pCJ-3/ME at distinct weight ratios formed complexes on the nanometer scale, with the exception of the weight ratio at 1.0:1.0. At this weight ratio, theoretical calculations demonstrated that the overall charge ratio (of the positively charged —NH₃⁺ groups on CS to the negatively charged —PO₄⁻ groups on DNA) of the chitosan nanoparticles was approximately 1.0:1.0, thus resulting in aggregation. The diameters of the prepared chitosan nanoparticles were in the range of 150-270 nm with a negatively or positively charged zeta potential, depending on the relative concentrations of CS and DNA used. When the amount of negatively charged DNA sufficiently exceeded that of positively charged CS (CS:DNA=0.5:1.0), the chitosan nanoparticles formed tended to have DNA exposed on their surfaces and thus had a negative surface charge. In contrast, when the amount of the positively charged CS significantly exceeded that of the negatively charged DNA (CS:DNA=1.5:1.0-5.0:1.0), some of the excessive CS molecules were arranged on the surfaces of the obtained chitosan nanoparticles. Thus, the resulting chitosan nanoparticles displayed a positive surface charge. The diameters of nanoparticles observed by scanning electron microscopy (SEM) were the same with those obtained by photon correlation spectroscopy (PCS).

Detection of Green Fluoresce Protein and JEV Envelope Protein Expression in Skin of C3H/HeN Mice with Transdermal Delivery by Low-Pressure Gene Gun

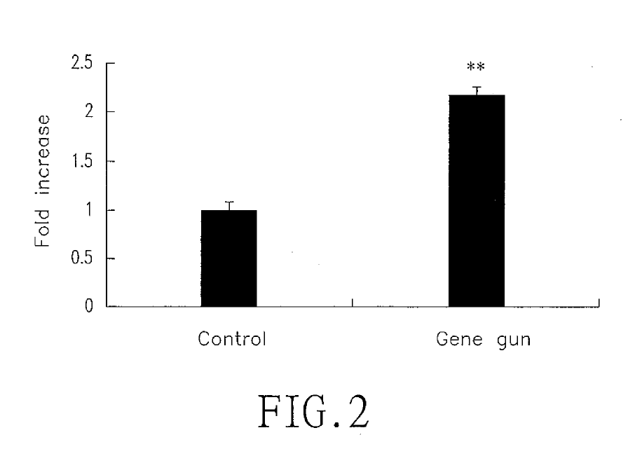

To detect green fluoresce protein and JEV envelope protein expression in skin of transdermal immunized C3H/HeN mice, groups of four 6-8 week-old female C3H/HeN mice were immunized with 50 μg of chitosan-DNA complexes by the low pressure gene gun. Mice inoculated with plasmid alone without coating chitosan were used as control. After 72 h, the skins were obtained and examined by fluorescence microscopy. Most of the GFP positive cells were located in the superficial epidermis area. Positive cells were also detected in hair follicles of epidermis and dermis under the site of topical application, but not in muscle. The efficiency of chitosan-DNA complexes was compared by delivering pCJ-3/ME to the mice skin with each vehicle and then measuring the expression of target protein in the treated skin. With reference to FIG. 2, JEV E protein expression in the low-pressure gene gun inoculation group was more than 2.2±0.05 times that of the group inoculated with no chitosan coated plasmid. From these data of the expression level of green fluorescent protein or envelop protein in the transdermally delivered mice skin by the low-pressure gene gun showed that chitosan-DNA complexes could be a DNA delivery vehicle for transdermal immunization.

Kinetics of GFP⁺ Cells and DC Cell Migration from Skin to Lymph Nodes in Transdermally Delivered Skin of C3H/HeN Mice

The epidermal predominant antigen presenting cell, Langerhans cell, has been shown to possess a constant level of transit from the skin to the draining lymph node (Glenn et al., Expert. Opin. Investig. Drugs 8:797-805 (1999); Garg et al., Nat. Immunol. 4:907-912 (2003)). First, an investigation was carried out for observing that Langerhans cell uptake the chitosan-DNA complexes and express in transdermal delivered skin of C3H/HeN mice. Thus, groups of three 6-week-old female C3H/HeN mice were shaved and hair removal cream was used to remove hair residues, then mice were immunized with 50 μg of chitosan/pGFP-N1 complexes by the low pressure gene gun. Then topical sites were collected in 72 hrs after transdermal delivery and immunofluorescent staining dendritic cells (DCs) from mice inoculated with pGFP-N1. GFP auto-fluorescence (in green under microscope) and the skin DCs were immunostained by PE-anti mouse CD11c (in red under microscope). How and rate of cell migration of Langerhans cell from skin to the lymph nodes in transdermal delivered skin of C3H/HeN mice were investigated. The C3H/HeN mice were immunized with 50 μg of chitosan/pCMVβ complexes by the low pressure gene gun. After 0, 24, 48, 72 and 96 hours, lymph nodes were collected and examined by flowcytometer and β -galactosidase staining. β-galactosidase could express in axilla and inguinal lymph node, furthermore major in inguinal lymph node, thus might a homing marker on lymphocyte due to different result (Anjuère et al., Blood 93:590-598 (1999); Xu et al., J. Exp. Med. 197:1255-1267). And the FACS analysis indicated that GFP positive cells could be detected until day 4. The GFP positive cells in inguinal lymph nodes were about 3%.

The Antibody Titer and the Protective Immunity in C3H/HeN Mice Provoked by Transdermally Immunized with Chitosan/pCJ-3/ME Complexes

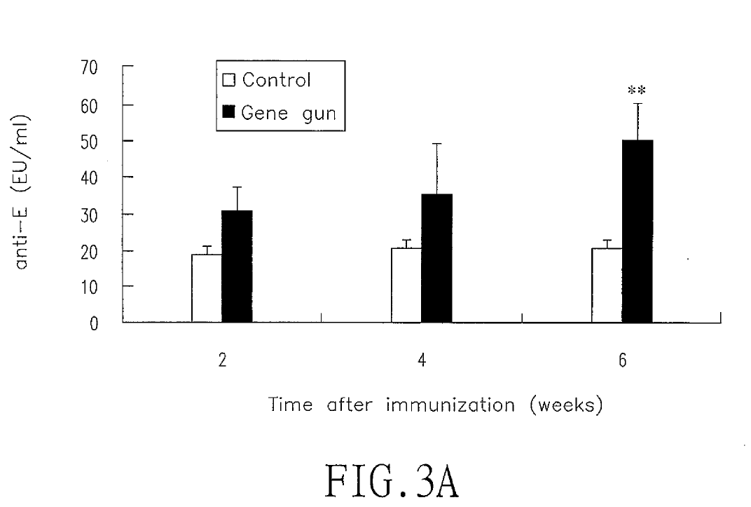

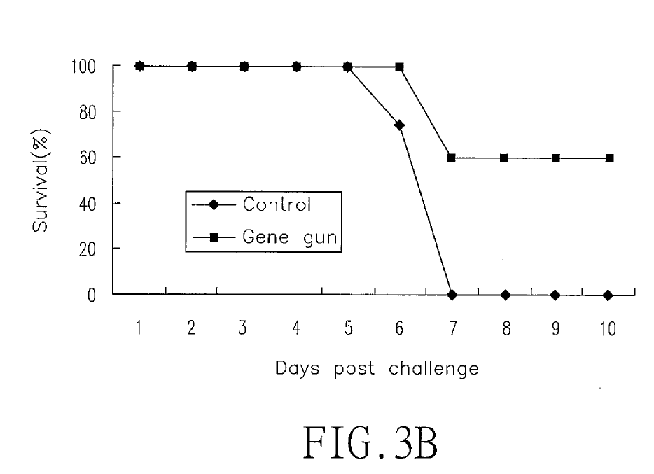

Transdermal delivery of JEV DNA vaccine by low pressure gene gun was less effective without chitosan coated. To determine whether in vivo transdermal delivery by low pressure gene gun could enhance the efficacy of chitosan coated JEV DNA vaccine, the abdomen skin of C3H/HeN mice were transdermal immunized with 50 μg of pCJ-3/ME DNA with or without coating chitosan, respectively. The plasmid alone without coating chitosan was used as a control. The mice received two boosters with the same amount of DNA after 2-week intervals using the same immunization method. As shown in FIGS. 3A, mice immunized by low pressure gene gun with plasmid pCJ-3/NE without coating chitosan did not produce any serum anti-E antibodies at week 8, whereas the group treated in the same way, but with chitosan coated, had high serum titers of E-specific antibody (50.1±10 U/ml). All mice were then challenged at week 8 with 50LD₅₀ (3×10⁷ PFU per mouse) of JFV Beijing-1. As shown in FIG. 3B, none of the mice in the control pCJ-3/ME without chitosan coated group survived the lethal challenge. In contrast, 60% of the low pressure gene gun with chitosan coated DNA vaccine group survived. These results show that JEV DNA vaccine given by tansdermally immunized with low pressure gene gun induces higher antibody titers and confers protection, against lethal JEV challenge.

The Isotypes of Specific Anti-E Antibody in C3H/HeN Mice Given Transdermal Immunization with Chitosan/pCJ-3/ME

Different inoculation approaches and routes of antigen can result in different antibody subclasses and different T helper (Th) cell types during the immune response. In general, Th1 immune responses promote the production of IgG2a antibody, whereas Th2 immune responses enhance the antibody production of IgG1. The IgG isotypes produced by tansdermally immunization with chitosan/pCJ-3/ME in C3H/HeN mice were analyzed. Similar titers of specific anti-E IgG were measured in both groups of immunized mice on day 28 after priming. Regarding the IgG subclass profiles, the tansdermally delivery groups produced almost exclusively IgG1 anti-E antibody, whereas IgG2a antibody was only detected in mouse with a lower titer.

### Example 2

### Immunization Against JEV by DNA Vaccine by Transcutaneous Delivery Using Skin Patch

### Preparation of Chitosan Nanoparticles

Chitosan nanoparticles were produced by ionic gelation of the positively charged CS with the TPP. As shown in Table 1, CS: pCJ-3/ME at distinct weight ratios formed complexes on the nanometer scale, with the exception of the weight ratio at 1.0:1.0. At this weight ratio, theoretical calculations demonstrated that the overall charge ratio (of the positively charged —NH₃⁺ groups on CS to the negatively charged —PO₄⁻ groups on DNA) of the chitosan nanoparticles was approximately 1.0:1.0, thus resulting in aggregation. The diameters of the prepared chitosan nanoparticles were in the range of 150-270 nm with a negatively or positively charged zeta potential, depending on the relative concentrations of CS and DNA used. When the amount of negatively charged DNA sufficiently exceeded that of positively charged CS (CS:DNA ) 0.5:1.0), the chitosan nanoparticles formed tended to have DNA exposed on their surfaces and thus had a negative surface charge. In contrast, when the amount of the positively charged CS significantly exceeded that of the negatively charged DNA (CS:DNA) 1.5:1.0-5.0:1.0), some of the excessive CS molecules were arranged on the surfaces of the obtained chitosan nanoparticles. Thus, the resulting chitosan nanoparticles displayed a positive surface charge (Table 1).

TABLE 1

| CS-DNA ratio (w/w) | Mean particle size (nm) | Zeta potential (mV) |
|---|---|---|
| 0.25 | 218.3 ± 10.18 | −50.62 ± 3.69 |
| 0.5 | 152.3 ± 1.94 | −29.12 ± 3.82 |
| 1 | 2830 ± 760.72 | 6.36 ± 2.31 |
| 1.5 | 212.6 ± 1.7 | 30.74 ± 3.51 |
| 2 | 204.8 ± 4.3 | 30.82 ± 0.64 |
| 2.5 | 224 ± 5.32 | 31.5 ± 1.29 |
| 3 | 244.2 ± 1.78 | 31.68 ± 2.4 |
| 3.5 | 210.8 ± 4.06 | 33.12 ± 0.59 |
| 4 | 226.2 ± 3.9 | 35.88 ± 2.71 |
| 4.5 | 228.2 ± 5.37 | 36.48 ± 0.41 |
| 5 | 263.9 ± 9.49 | 35.84 ± 0.15 |

### Cell Viability Studies

In order to estimate whether the preparation process of the particles would induce any cytotoxicity, the cell viability of BHK-21 and Vero cells after treatment with different NIP ratio of chitosan-DNA nanoparticles complexes, at different concentrations, was evaluated by MTS assay. No cytotoxicity was observed for the particle suspensions and a cell viability of around 80% was observed in all test groups. The proliferating cells are metabolically more active than non-proliferating (resting) cells, these results can also be interpreted as the possible impact of chitosan nanoparticles on cell proliferation. This is probably an indication that the chitosan nanoparticles may favorably influence lysosomal and mitochondrial activity of the cells (Glenn et al., Infect Immun 67(3):1100-6 (1999)).

### Transfection Efficacy of Chitosan DNA Complexes

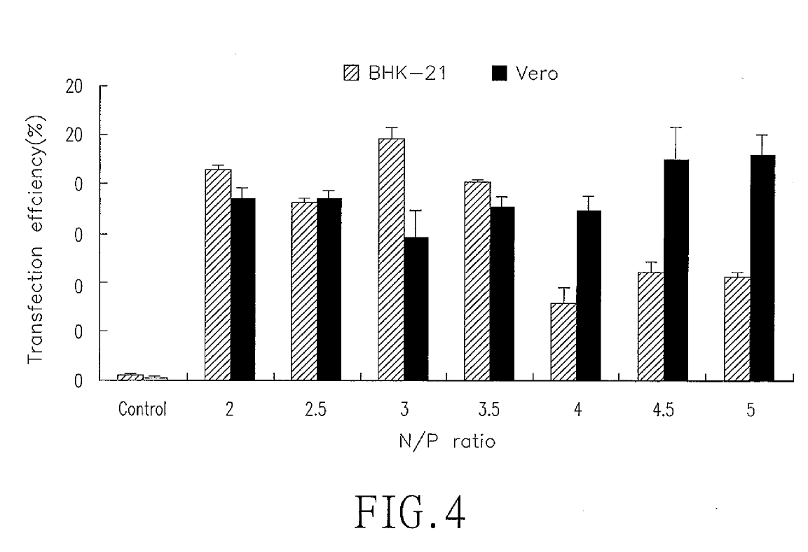

Transfection of BHK-21 or Vero cells using chitosan DNA complexes was made in the presence of 10% serum, and fluorescence was monitored over a period of 24 to 48 h after the transfection step. In BHK-21 cells, the fluorescence intensity was clearly dependent on the post-transfection time, with maximal gene expression reached at 48 h (FIG. 4). The flow cytometry results showed that the highest transfection activity of CS-DNA complexes in BHK-21 and Vero cells were made with N/P ratio=3 and 5, respectively. These high transfection efficiencies of the chitosan/pGFP-N1 complexes are considered to be due to the cell uptake and intracellular tracking of the complexes. These results suggest that the intracellular trafficking of complexes after cell uptake has strong influences on the transfection efficiency. Therefore, the mechanism on the transfection with the CS-DNA complexes was investigated using fluorescence labeled plasmid or chitosan.

Detection of JEV Envelope Protein Expression in Skin of C3H/HeN Mice with Transcutaneous Delivery by Skin Patch

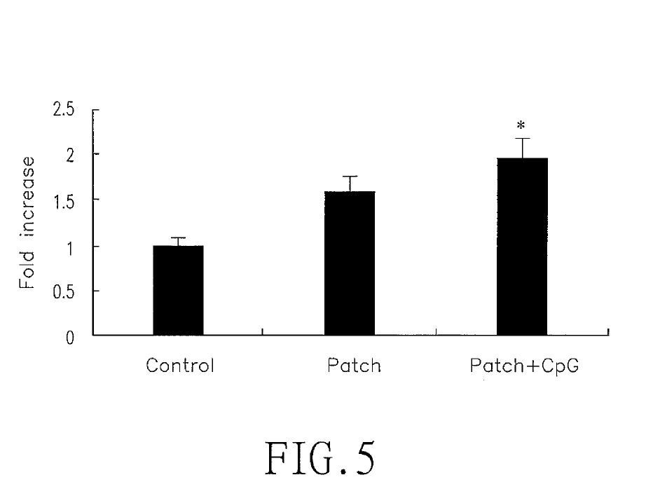

To detect JEV-E protein expression in vivo, groups of five 6˜8 week-old female C3H/HeN mice were transcutaneous inoculated with 50 μg of chitosan/pCJ-3/ME complexes by skin patch. The skin patch (100 μl in volume, CS-DNA complexes containing 50 μg of pCJ-3/ME or pGFP-N1in sterile DI water within non-woven fabrics) was transcutaneously delivered on a 1 cm² area of the hairless dorsal back skin. Mice inoculated with plasmid alone without coating chitosan were used as controls. After 72 h, the mice skins were obtained and the JEV-E protein expression of each sample was detected by dot-blot assay. The JEV-E protein expression in the skin inoculated with CS-DNA complexes by skin patch group was more than 1.5 times that of the group inoculated with plasmid alone (FIGS. 5). It has been reported that bacterial DNA or synthetic oligodeoxyribonucleotides (ODN) containing CpG motif stimulate the immune systems of mice and humans. The CpG motif-containing ODN has potent adjuvant activity by activating macrophages and natural killer (NK) cells in several mouse models (Verthelyi et al., J Immunol 168: 1659-1663 (2002)). FIG. 5 show that the mice inoculated the skin patch with CS-DNA complexes combined CpG motif-containing ODN can induce the highest JEV-E protein expression. From these data of the expression level of JEV-E protein in the transcutaneously delivered mice skin by skin patch showed that chitosan-DNA complexes could be the DNA delivery vehicle in transcutaneous immunization.

The Antibody Titer and the Protective Immunity in C3H/HeN Mice Provoked by Transcutaneously Immunized with Chitosan/pCJ-3/ME Complexes

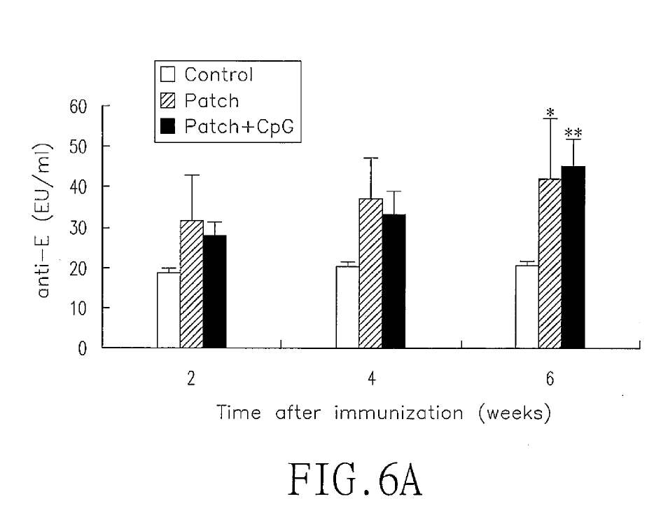

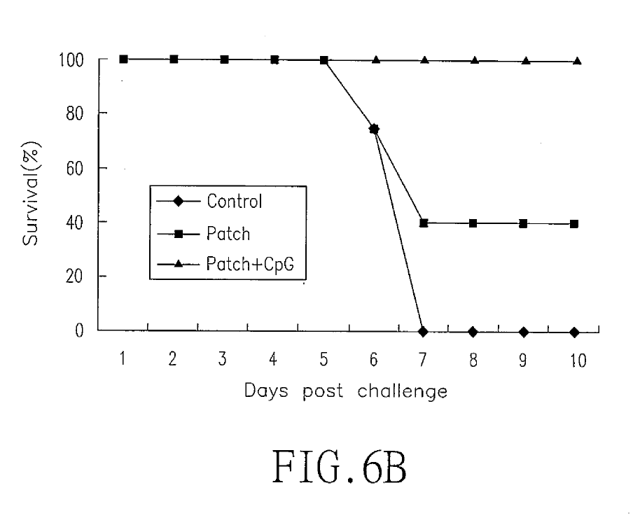

JEV DNA vaccine by transcutaneous delivery using skin patch was less effective without chitosan coating. To determine whether in vivo transcutaneous delivery by skin patch enhanced the efficacy of chitosan coated JEV DNA vaccine, the abdomen skin of C3H/HeN mice were transcutaneously immunized with 50 μg of pCJ-3/ME DNA with or without chitosan coating, respectively. The plasmid alone without coating chitosan was used as a control. The mice received two boosters with the same amount of DNA at 2-week intervals using the same immunization method. With reference to FIG. 6A, mice immunized by skin patch with plasmid pCJ-3/ME without coating chitosan did not produce any serum anti-E antibodies at week 8, whereas the group treated in the same way, but with chitosan coated, had high serum titers of E-specific antibody (42.1±15 U/ml). FIG. 6A also show that the mice inoculated the skin patch with CS-DNA complexes combined CpG motif-containing ODN can induce the highest serum titers of E-specific antibody (45.2±6.4 U/ml). All mice were then challenged at week 8 with 50LD₅₀ (3×10⁷ PFU per mouse) of JEV Beijing-1. As shown in FIG. 6B, none of the mice in the control pCJ-3/ME without chitosan coated group survived the lethal challenge. In contrast, 40% of the skin patch with chitosan coated DNA vaccine group survived. The mice inoculated the skin patch with CS-DNA complexes combined CpG motif-containing ODN have 100% survival rate. The CpG motif-containing ODN may be a potent adjuvant for DNA vaccine given by transcutaneous delivery with skin patch. These results show that JEV DNA vaccine given by transcutaneous delivery with skin patch induces higher antibody titers and confers protection, against lethal JEV challenge.

The Isotypes of Specific Anti-E antibody in C3H/HeN Mice Immunized by Transcutaneous Delivery of Chitosan/pCJ-3/ME

Different inoculation approaches and routes of antigen can result in different antibody subclasses and different T helper (Th) cell types during the immune response. In general, Th1 immune responses promote the production of IgG2a antibody, whereas Th2 immune responses enhance the antibody production of IgG1. The IgG isotypes produced by transcutaneous immunization with chitosan/pCJ-3/ME in C3H/HeN mice were analyzed. Similar titers of specific anti-E IgG were measured in both groups of immunized mice on day 28 after priming. Regarding the IgG subclass profiles, the transcutaneous delivery groups produced almost exclusively IgG1 anti-E antibody, whereas IgG2a antibody was only detected in mouse with a lower titer.

### Example 3

### CS-Collagen Complexes Transdermally Delivered by a Low-Pressure Gene Gun and Transcutaneously Delivered by Skin Patch

### Preparation of CS-Collagen Complexes

Chitosan nanoparticles were produced by ionic gelation of the positively charged CS with the TPP. A sample of Ig of collagen was dissolved in 10 mL deionized (DI) water (pH 6.0). The collagen solution was then diluted with DI water to make a 1 mg/mL collagen stock solution. The collagen stock solution (1 mL) was premixed with aqueous chitosan nanoparticles (50 μg/mL, 2 mL) under magnetic stirring at room temperature.

### Cell Viability Studies

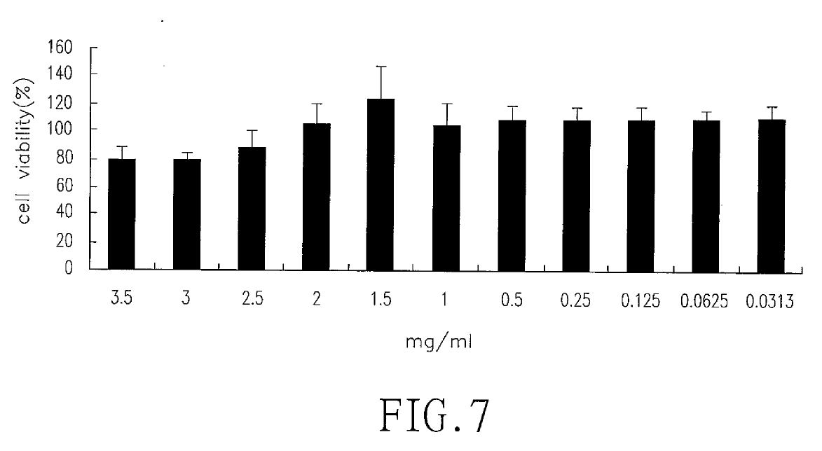

In order to estimate whether the preparation process of the particles would introduce any cytotoxicity, BHK-21 cells were treated with different concentrations of chitosan-collagen complexes and the cell viability was evaluated by MTS assay. FIG. 7 shows that no cytotoxicity effects were observed for the chitosan nanoparticle suspensions. A cell viability of around 80% was observed in all groups. Proliferating cells are metabolically more active than non-proliferating (resting) cells. Thus, these results indicated a possible impact rather than cellular toxicity of chitosan-collagen complexes on cell proliferation. These results showed that chitosan-collagen complexes show good in vitro biocompatibility and support proliferation and normal functions of fibroblasts. (Chen et al., colloids and surfaces A. 313-314:183-8 (2008)).

### NO (Nitric Oxide) Assay of CS-Collagen Complexes in RAW 264.7

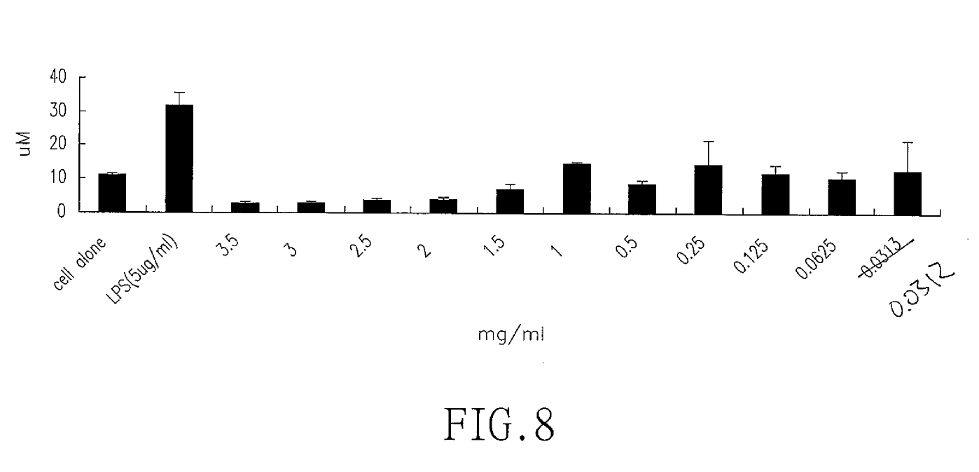

In order to estimate whether the chitosan-collagen complexes would induce any inflammation, the macrophage cells (RAW 264.7) were treated with different concentrations of chitosan-collagen complexes and the nitrite accumulate was evaluated by NO (nitric oxide) assay. Because of its instability in physiological solutions, most of NO is rapidly converted to nitrite (NO₂⁻) and further to nitrate (NO₃⁻). Briefly, nitrate was converted to nitrite with aspergillus nitrite reductase, and the total level of nitrite was determined with the Griess reagent. The absorbance was determined at 550 nm with ELISA reader. FIG. 8 shows that the inflammation indexes (nitrite concentration) were significantly induced by LPS treatment as positive control, but chitosan-collagen complexes treatment groups were not induced higher nitrite accumulation like cell without any treatment. The data show that chitosan-collagen complexes do not induce inflammation and they are fit for the cosmetic use.

Transdermal and Transcutaneous Delivery of FITC-Labeled CS-Collagen Complexes Visualized with Fluorescence Microscope

In order to estimate whether the chitosan-collagen complexes could be transdermally delivered by a low-pressure gene gun and transcutaneously delivered by skin patch, the FITC-labeled CS-collagen in skin of transdermal immunized C3H/HeN mice were evaluated by fluorescence microscope. The synthesis of the FITC-labeled collagen was based on the reaction between the isothiocyanate group of FITC and the primary amino groups of collagen as reported in the literature. (Lim et al., Pharm. Res. 20, 1812-19 (2003)). To remove the unconjugated FITC, the precipitate was subjected to repeated cycles of washing and centrifugation (10000 g for 10 min) until no fluorescence was detected in the supernatant. The obtained FITC-labeled collagen was used to prepare the CS-protein complexes.

Female C3H/HeN mice (6-8 weeks of age) were used for transdermal and transcutaneous delivery There were 6 mice in each group (3 mice were used for transcutaneous delivery studies with skin patch, the other 3 mice were used for transdermal delivery studies with a low-pressure gene gun). The C3H/HeN mice were anesthetized using acepromazine maleate (i.p.) and hair covering a restricted area of abdomen skin was removed with a shaver. The shaved skin was further treated with a depilatory (Yanagiya, Japan) which potentially facilitates removal of more cornified epithelium. Subsequently, CS-protein complexes containing 25 μg FITC-collagen in sterile DI water (100 μl) were loaded in a low-pressure gene gun and bombarded into the skin (Nitrogen pressure setting of 60 psi) of 3 mice in a group. The skin patch (100 μl in volume, CS-protein complexes containing 25 μg FITC-collagen in sterile DI water within non-woven fabrics) was transcutaneously delivered on a 1 cm² area of the hairless dorsal back skin of the each other 3 mice. One hour later, mice topically applied with CS-collagen complexes were sacrificed. The skin of the mice were collected and immediately frozen to −20° C. The tissue were then frozen in O.C.T. embedding medium (Sakura Finetek USA, Torrance, Calif.) and then cut into thin sections. The sections were visualized under the Olympus fluorescence microscopy BX-51 (Olympus, Janpen) equipped with a digital camera. The fluorescence images were captured by digital camera and the intensity was quantified by the Quantity One® (Bio-Rad, Calif., USA).

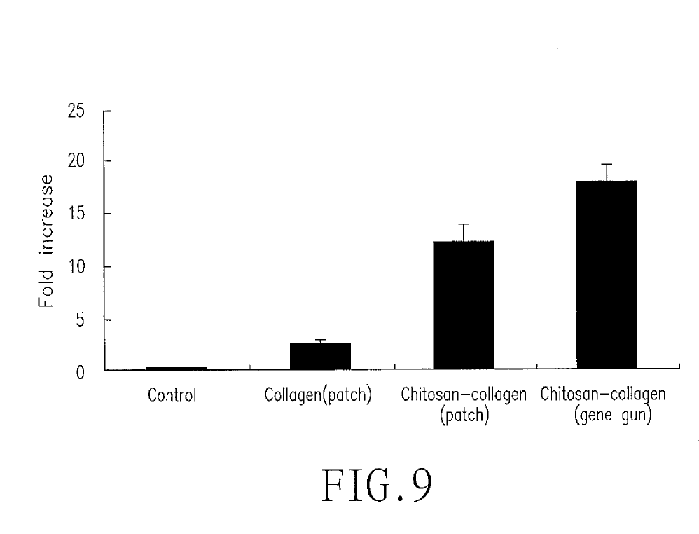

In each group of FITC-labeled CS-collagen complexes transdermally delivered by a low-pressure gene gun and transcutaneously delivered by skin patch, they were observed that most of the green fluorescence signals were located in the superficial epidermis area and derma but not in muscle of mice having. The fluorescence intensity in the skin inoculated with FITC-labeled CS-collagen complexes by skin patch group was more than 13 times that of the group inoculated with FITC-labeled collagen alone (FIGS. 9). The fluorescence intensity in the skin inoculated with FITC-labeled CS-collagen complexes by low-pressure gene gun group was higher than 18 times that of the group inoculated with FITC-labeled collagen alone (FIGS. 9). The lowest fluorescence intensity in the skin was measured in control mice without coating chitosan. From these data of fluorescence intensity in the transcutaneously delivered mice skin by skin patch or transdermally delivered by a low-pressure gene gun showed that chitosan-protein complexes could be the protein delivery vehicle in transcutaneous or transdermally delivery system.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A chitosan vehicle comprising chitosan (CS) nanoparticles being at least 81% deacetylated.

2. A method for making a chitosan vehicle comprising:
dissolving a chitosan to a concentration of 1% (w/v) in 167 mM acetic acid solution to generate a chitosan solution;
adjusting the chitosan solution to pH 4.0-6.0;
adding a crosslinking agent into the chitosan solution to generate a mixture; and
degrading the chitosan solution to generate chitosan nanoparticles.

3. The method as claimed in claim 2, wherein the chitosan solution is degraded with a method selected from a group consisting of ionic gelation, emulsion polymerization and emulsification-diffusion.

4. A CS-DNA (chitosan-deoxyribonucleic acid) complex comprising
a chitosan vehicle having
chitosan nanoparticles having
amino groups;
a crosslinking agent; and
a DNA crosslinked to the chitosan nanoparticles and having
phosphate anions, wherein a ratio of the phosphate anions to the amino groups is 1:0.25 to 1:5.

5. The CS-DNA complex as claimed in claim 4, wherein the crosslinking agent is TPP.

6. The CS-DNA complex as claimed in claim 4, wherein the ratio of the phosphate anions to the amino groups is 1:2 to 1:5.

7. The CS-DNA complex as claimed in claim 4, wherein the DNA is a DNA plasmid.

8. The CS-DNA complex as claimed in claim 4, wherein the DNA is a DNA vaccine.

9. The CS-DNA complex as claimed in claim 8, wherein the DNA is a Japanese Encephalitis Virus (JEV) DNA vaccine.

10. The CS-DNA complex as claimed in claim 8 farther comprising an adjuvant.

11. The CS-DNA complex as claimed in claim 10, wherein the adjuvant is an oligodeoxyribonucleotides (ODN) containing CpG motif.

12. A method for making a CS-DNA complex comprising:
preparing a chitosan vehicle having chitosan nanoparticles;
mixing a DNA with the chitosan vehicle; and
adding a crosslinking agent allowing the DNA to be crosslinked to the chitosan nanoparticles.

13. The method as claimed in claim 12, wherein the chitosan vehicle is made from a chitosan solution of pH 5.5.

14. A CS-protein complex comprising:
a chitosan vehicle having chitosan nanoparticles;
a crosslinking agent; and
a protein crosslinked and encapsulated inside the chitosan nanoparticles of the chitosan vehicle, wherein a ratio of the crosslinking agent to the chitosan nanoparticles is from 1:0.5 to 1:5.

15. The CS-protein complex as claimed in claim 14, wherein the crosslinking agent is TPP.

16. The CS-protein complex as claimed in claim 14, wherein the protein is collagen.

17. The CS-protein complex as claimed in claim 14, wherein the protein is epidermal growth factor (EGF).

18. A method for making a CS-protein complex comprising:
dissolving a chitosan to an acetic acid solution under gentle heat to generate a chitosan solution;
adjusting the chitosan solution to pH 6;
degrading the chitosan solution to generate chitosan nanoparticles in the chitosan solution;
centrifuging the chitosan solution at 12,000 g and removing pelleted particles;
diluting the chitosan nanoparticles solution with water to make an aqueous chitosan solution;
preparing a protein stock solution comprising a protein;
mixing the protein stock solution with the aqueous chitosan solution at room temperature to generate a mixture solution comprising a mixture of the protein and chitosan nanoparticles;
allowing the mixture solution to react for a period of time at room temperature;
centrifuging the mixture solution to generate a supernatant having a CS-protein complex;
obtaining the CS-protein complex.

19. The method as claimed in claim 18, wherein the chitosan vehicle is made from a chitosan solution of pH 6.0.

20. The method as claimed in claim 18, wherein the ratio of the protein to the chitosan nanoparticles is 1:2.

21. A gene gun projectile comprising a CS-DNA complex of claim 4.

22. A gene gun projectile comprising a CS-protein complex of claim 14.

23. A skin patch comprising a CS-DNA complex of claim 4.

24. A skin patch comprising a CS-protein complex of claim 14.

25. An ointment comprising a CS-DNA complex of claim 4.

26. An ointment comprising a CS-protein complex of claim 14.
